(19) United States
(12) Patent Application Publication
(10) Pub. No.: US 2016/0331775 A1
(43) Pub. Date: Nov. 17, 2016

(54) E-SELECTIN INHIBITION WORKS IN COMBINATION WITH LOW-MOLECULAR WEIGHT HEPARIN TO DECREASE VENOUS THROMBOSIS AND BLEEDING RISK

(71) Applicants: THE REGENTS OF THE UNIVERSITY OF MICHIGAN, ANN ARBOR, MI (US); GLYCOMIMETICS, INC., ROCKVILLE, MD (US)

(72) Inventors: DANIEL D. MYERS, JR., Ann Arbor, MI (US); THOMAS W. WAKEFIELD, Ann Arbor, MI (US); SUMAN SOOD, Ann Arbor, MI (US); JOHN L. MAGNANI, Gaithersburg, MD (US)

(21) Appl. No.: 15/154,308

(22) Filed: May 13, 2016

Related U.S. Application Data
(60) Provisional application No. 62/161,357, filed on May 14, 2015.

(51) Int. Cl. A61K 31/727; A61K 31/7056; A61K 31/7048; A61K 31/7034

(52) U.S. Cl. CPC A61K 31/727; A61K 31/7034; A61K 31/7056; A61K 31/7048

(57) ABSTRACT

The present disclosure provides compositions and methods for treating and preventing thrombosis. In particular, the present disclosure provides methods and compositions for treating and preventing thrombosis utilizing E-selectin inhibition in combination with heparin.

### CROSS REFERENCE TO RELATED APPLICATIONS

This application claims the benefit of U.S. provisional application Ser. No. 62/161,357, filed May 14, 2015, which is incorporated herein by reference in its entirety.

### STATEMENT REGARDING FEDERALLY SPONSORED RESEARCH OR DEVELOPMENT

This application was supported by Grant No. HHSN268201400012C awarded by the National Institutes of Health. The government has certain rights in the invention.

### FIELD OF THE DISCLOSURE

The present disclosure provides compositions and methods for treating and preventing thrombosis. In particular, the present disclosure provides methods and compositions for treating and preventing thrombosis utilizing E-selectin inhibition in combination with heparin.

### BACKGROUND

There remains an unmet medical need for a translatable therapeutic that can treat venous thrombosis (VT) in combination with lower, safer levels of low molecular weight heparin (LMWH) anticoagulation.

### BRIEF DESCRIPTION OF DRAWINGS

FIG. 1A shows that GMI-1271 works in combination with LMWH to decrease venous thrombosis. FIG. 1B shows that GMI-1271 does not increase bleeding potential.

### SUMMARY OF THE DISCLOSURE

The present disclosure provides compositions and methods for treating and preventing thrombosis. In particular, the present disclosure provides methods and compositions for treating and preventing thrombosis utilizing E-selectin inhibition in combination with heparin.

The described methods provide the further advantage of decreasing the risk of bleeding associated with treatment with heparin.

E-selectin antagonism can decrease thrombus formation and associated inflammation. E-selectin (CD-62E) is a cell adhesion molecule that is expressed on activated endothelial cells and plays an important role in leukocyte recruitment to the site of vascular injury. Exemplary E-selectin inhibitors (e.g., those described herein such as GMI-1271) are designed to mimic the bioactive conformation of the sialyl-Le′ carbohydrate binding domain of E-selectin and are specific E-selectin inhibitors. Using a well characterized mouse model of venous thrombosis it was demonstrated that E-selectin antagonism with GMI-1271 in combination with either fractionated or low-molecular weight heparin (LMWH) anticoagulation, could significantly decrease venous thrombosis (VT) and decrease the risk of elevated bleeding, while significantly decreasing fibrin deposition in mouse venous thrombi.

In some embodiments, the present disclosure provides combination therapy comprising E-selectin antagonism combined with fractionated or LMWH permitting lower doses of these standard anticoagulants for the treatment and prevention of VT. Lower doses significantly decreases bleeding risks associated with anticoagulation. This has a tremendous therapeutic advantage over current therapies that present bleeding risk in the clinics.

The present disclosure is not limited to particular E-selectin antagonists. In some embodiments, antagonists are glycomimetic compounds described, for example, in WO 2013/096926, herein incorporated by reference in its entirety. In general, the amount of a glycomimetic compound described herein, that is present in a dose, ranges from about 0.01 μg to about 1000 μg (e.g., 0.05 to 900 μg, 0.1 to 500 μg, 0.5 to 500 μg, 1.0 to 500 μg, 5.0 to 500 μg, 10 to 250 μg, 50 to 250 μg, 10 to 100 μg, etc.) per kg weight of the host.

For example, in some embodiments, the present disclosure provides a method of reducing and/or preventing thrombosis, comprising: administering an E-selectin antagonist and heparin including fractionated or low-molecular weight heparin (LMWH) to a subject diagnosed with or at risk of thrombosis (e.g., venous thrombosis). In some embodiments, the E-selectin antagonist is a glycomimetic (e.g., GMI-1271). Exemplary dosages of heparin include, but are not limited to, 0.1-20 mg/kg (e.g., 0.1-10, 0.1-5, 1-5, or 3-6 mg/kg or lower). In some embodiments, the method results in a decreased risk of elevated bleeding.

In some embodiments, the E-selectin antagonist is one or more of:

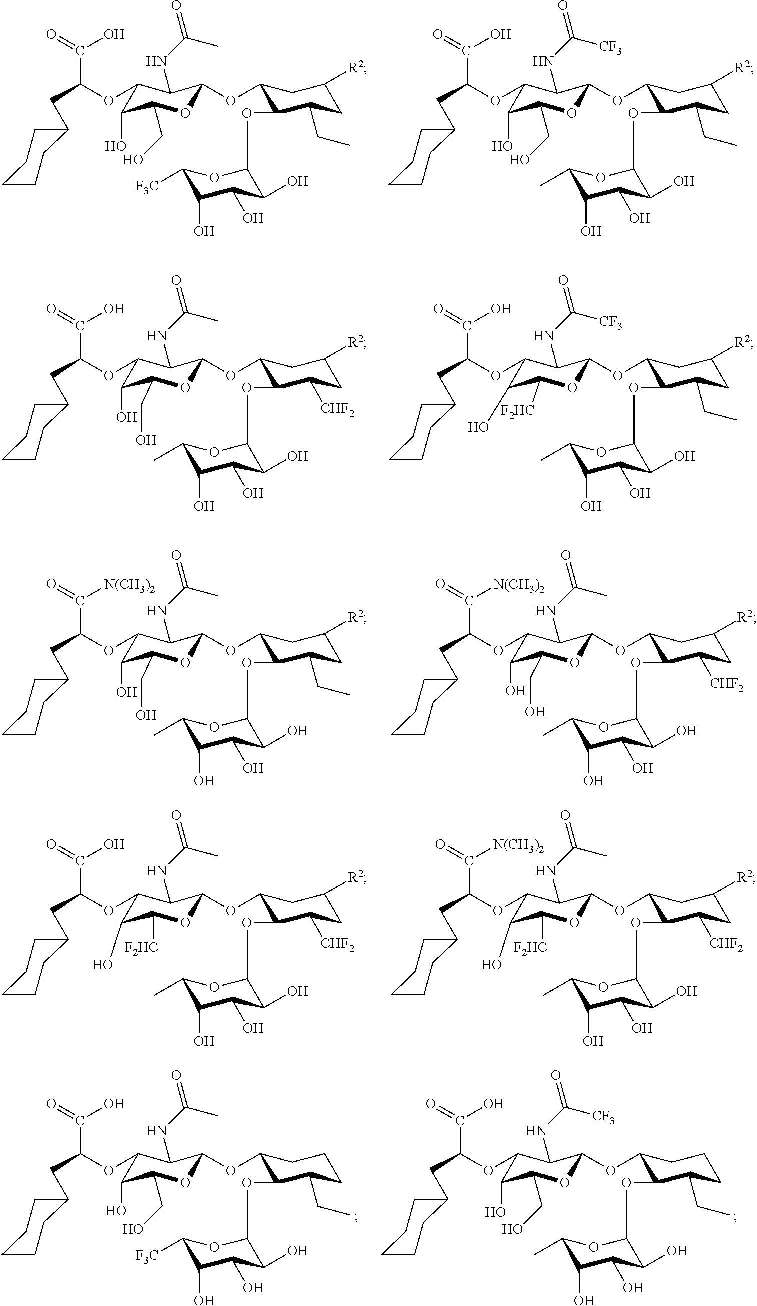

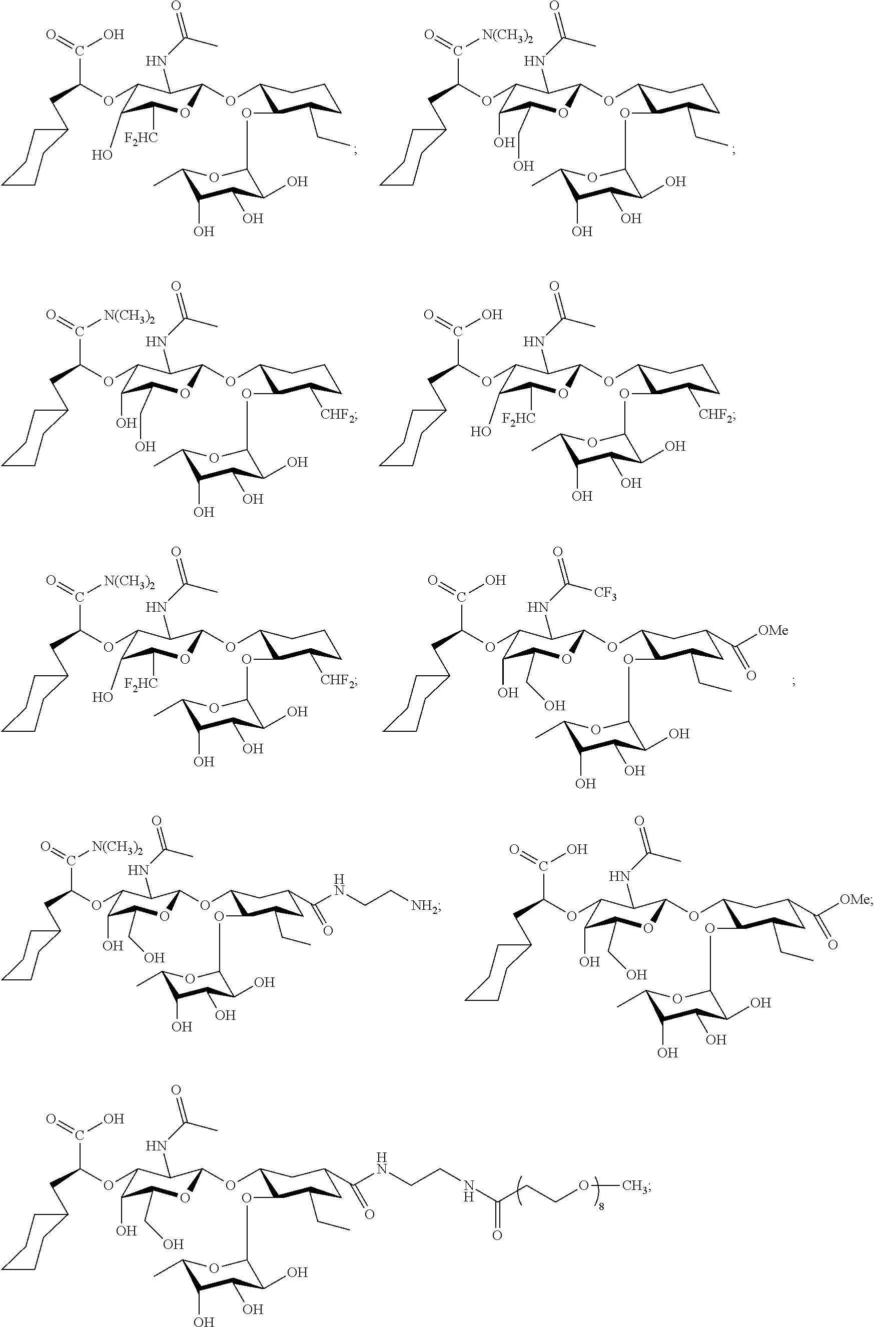

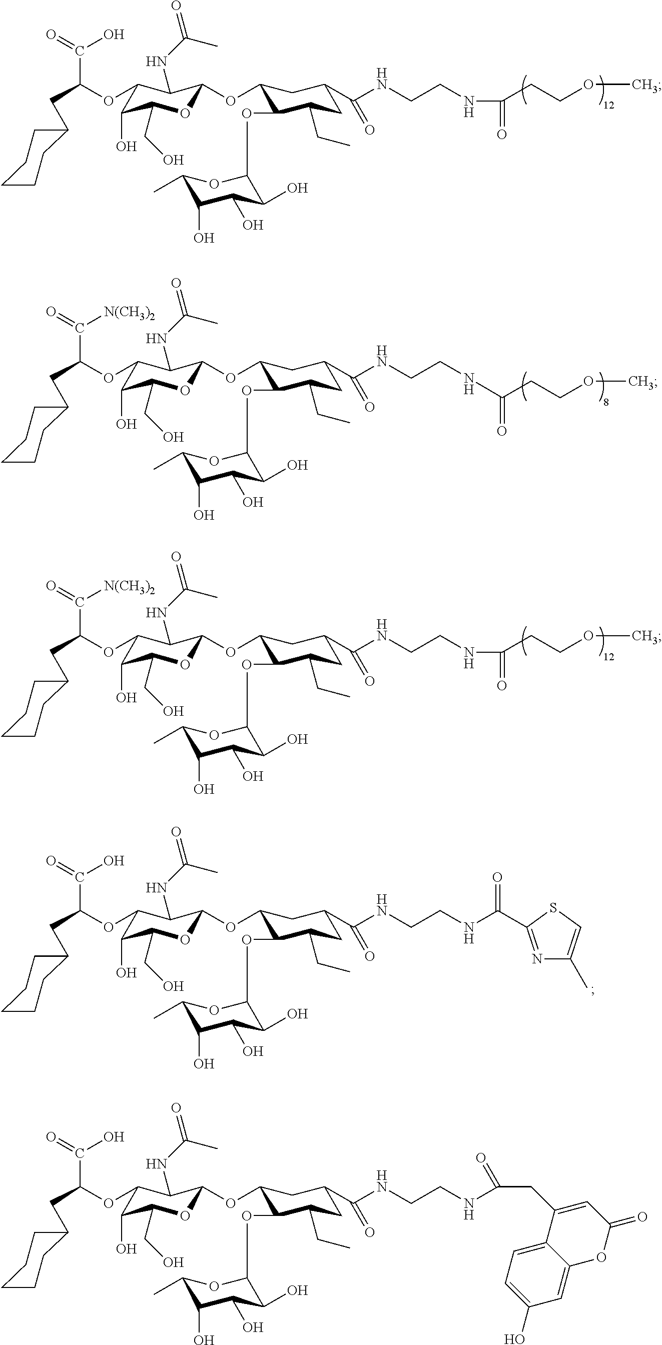

Further embodiments provide the use of an E-selectin antagonist in the treatment and/or prevention of thrombosis.

Additional embodiments provide a pharmaceutical composition comprising an E-selectin antagonist and heparin.

Additional embodiments are described herein.

### DESCRIPTION OF THE DISCLOSURE

The present disclosure provides compositions and methods for treating and preventing thrombosis. In particular, the present disclosure provides methods and compositions for treating and preventing thrombosis utilizing E-selectin inhibition in combination with heparin.

### Methods:

Male C57BL/6J mice, 10 weeks old (23-28 grams, n5), underwent our electrolytic IVC model (EIM) to produce a non-occlusive thrombosis, via electrical free radical stimulation (250 μAmp) for 15 minutes. Experimental groups included non-treated controls (CTR-No Tx), animals given LMWH (3-6 mg/kg, SQ, once daily (qd), the E-selectin inhibitor GMI-1271 20/kg intraperitoneal (IP) twice daily (BID), and a combination of the agents. The dose range of LMWH that produced anti-Xa levels in the therapeutic range (0.5-1.0 IU/mL) and significantly decreased thrombus weight in this mouse VT model was 5 and 6 mg/kg. Treated mouse groups received the first dose of experimental therapy immediately following thrombus induction and through day 2. Animals were euthanized 2 days post-thrombosis for tissue harvest and blood collection for the following evaluations: thrombus weight (grams), anti-Xa testing, and tail vein bleeding time (seconds).

### Results:

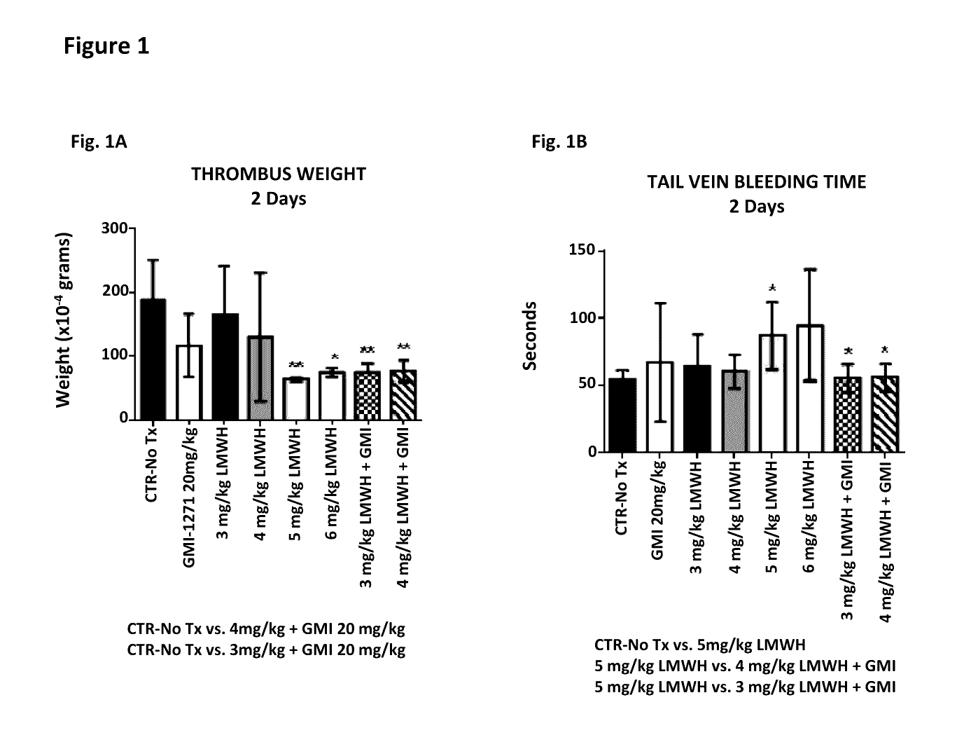

GMI-1271 Works in Combination with LMWH to Decreases Venous Thrombosis: LMWH dosed at 6 mg/kg and 5 mg/kg alone, significantly decreased venous thrombus weight at 2 days, versus non-treated controls (73.0±7.5, 62.8±1.9 vs. 186.8±63.9×10⁻⁴ grams, P<0.01). Notably, the combination of GMI-1271 (See e.g., WO 2013/096926; herein incorporated by reference in its entirety) 20 mg/kg+LMWH 4 mg/kg (33% dose decrease from LMWH 6 mg/kg), and GMI-1271 20 mg/kg+LMWH 3 mg/kg (50% dose decrease from LMWH 6 mg/kg dose), significantly decreased thrombus weight, versus non-treated controls (75.6±17.1, 73.0±14.8 vs. 186.8±63.9×10⁻⁴ grams, P<0.01), equivalent to the results of higher doses of LMWH alone (FIG. 1 A).

### GMI-1271 Does not Increase Bleeding Potential:

GMI-1271 administration did not significantly elevate tail bleeding times, versus non-treated control mice (67.00±44 vs. 53.8±7.5 seconds). LMWH dosed at 5 mg/kg and 6 mg/kg, elevated tail bleeding times in mice with the 5 mg/kg LMWH group significant versus non-treated controls (87±25 vs. 53.8±7.5 seconds, P<0.005). Both animal groups treated with GMI-1271, in combination with lower dose LMWH therapy, had tail bleeding times comparable to non-treated control animals (FIG. 1 B).

### Conclusion:

We report, for the first time, that GMI-1271 works in combination with LMWH to significantly reduce acute VT without increasing bleeding times and by inference, bleeding potential. E-selectin inhibition with GMI-1271 may be used to treat VT alone, or in combination with lower and safer levels of LMWH anticoagulation.

1. A method of reducing and/or preventing thrombosis, comprising:
administering to a subject in need thereof an effective amount of at least one E-selectin antagonist and heparin.

2. The method of claim 1, wherein the at least one E-selectin antagonist is a glycomimetic.

3. The method of claim 2, wherein said glycomimetic is GMI-1271.

4. The method of claim 1, wherein said heparin is fractionated or low-molecular weight heparin (LMWH).

5. The method of claim 1, wherein said thrombosis is venous thrombosis.

6. The method of claim 1, wherein said method results in a decreased risk of elevated bleeding.

7-12. (canceled)

13. A pharmaceutical composition comprising at least one E-selectin antagonist and heparin.

14. The composition of claim 13, wherein the at least one E-selectin antagonist is a glycomimetic.

15. The composition of claim 13, wherein said glycomimetic is GMI-1271.

16. The composition of claim 13, wherein said heparin is fractionated or low-molecular weight heparin (LMWH).
